(19) United States
(12) Patent Application Publication
(10) Pub. No.: US 2012/0149098 A1
(43) Pub. Date: Jun. 14, 2012

(54) EXTRACTION OF PROCESS FOR MESENCHYMAL STROMAL STEM CELLS

(75) Inventor: Erica MOLINO, Magliano Alfieri (Cuneo) (IT)

(73) Assignee: Davide VANNONI, Moncalieri (Torino) (IT)

(21) Appl. No.: 12/964,938

(22) Filed: Dec. 10, 2010

(51) Int. Cl. C12N 5/077

(52) U.S. Cl. 435/325

(57) ABSTRACT

Mesenchymal stromal stem cells are obtained starting with a biopsy sample from the iliac crest of the pelvis that is fragmented with surgical forceps in a saline solution and the saline solution containing the pulverised biopsy sample is then subjected to centrifugation, so to obtain mesenchymal stromal stem cells. The mesenchymal stromal stem cells are then selected by adhesion.

### FIELD OF THE INVENTION

The present invention concerns a procedure for extracting mesenchymal stromal stem cells.

### TECHNICAL BACKGROUND

Mesenchymal stromal stem cells are present in the medullary stroma [1]. They are pluripotent cells that, if appropriately directed, have the capacity to replicate and differentiate both in vivo and in vitro into various cell types such as osteoblasts, chondrocytes [2], myocytes [3], neuronal cells [4], to cite only a few examples. This differentiation potential makes mesenchymal stromal stem cells an important therapeutic resource for diverse pathologies [5].

In general practice mesenchymal stromal stem cells are obtained from bone marrow aspirates or by means of blood sampling. However, these samples are contaminated with other types of cells, such as for example, haematopoietic and/or endothelial cells, which represent an impediment to the attachment and expansion of mesenchymal stromal stem cells and a risk for microbiological contamination. In addition, aspirated samples contain a much lower absolute number of mesenchymal stromal stem cells with respect to those obtained by means of the extraction procedure object of the present invention.

### SUMMARY OF THE INVENTION

Therefore, considering these preambles, the need is felt for improving solutions for obtaining mesenchymal stromal stem cells.

The object of the present description is to provide such improving solutions.

According to the invention, said objective is obtained by means of the solution specifically recalled in the attached claims, which constitute an integral part of the present description.

The present invention concerns a procedure for extracting mesenchymal stromal stem cells from a biopsy sample of the iliac crest of the pelvis, obtaining mesenchymal stromal stem cells that are not contaminated by haematopoietic and/or endothelial cells. In particular, the procedure for extracting mesenchymal stromal stem cells envisions fragmentation of the biopsy sample in a solution, preferably a saline solution, and the successive centrifugation of the solution in order to obtain mesenchymal stromal stem cells, in particular in the form of a pellet.

### DETAILED DESCRIPTION OF SOME EMBODIMENTS

The invention will now be described in detail, by way of example only.

In the description that follows, numerous specific details are presented to provide complete comprehension of the embodiments. The embodiments can be practiced without one or more of the specific details, or with other methods, components, materials, etc. In other instances, well-known structures, materials or operations are not shown or described in detail to avoid obscuring certain aspects of the embodiments.

Reference throughout the present specification to “one embodiment” or “an embodiment” means that a particular feature, structure or characteristic described in connection with the embodiment is included in at least one embodiment. Thus, the phrase “in one embodiment” or “in an embodiment” in various places throughout the present specification are not necessarily all referring to the same embodiment. Furthermore, the details of features, structures, or characteristics may be combined in any suitable manner in one or more embodiments.

The headings provided herein are for convenience only and do not interpret the scope or meaning of the embodiments.

The standard techniques for extracting stem cells essentially envision removal of marrow blood from the spine or iliac crest by means of a large needle with drill. The drill avoids that bone stroma enters in the needle during the insertion and blocks it. In this way a cellular suspension constituted by (CD34+) haematopoietic stem cells, in addition to red blood cells and nucleated cells derived from the peripheral blood is obtained. A small fraction (less than 0.01%) is constituted of mesenchymal stem cells.

The present invention envisions an innovative method for extracting mesenchymal stromal stem cells.

Specifically, the procedure for obtaining mesenchymal stromal stem cells comprises:
obtaining a biopsy sample from the iliac crest of the pelvis of a patient,
fragmenting the biopsy sample with a surgical forceps, preferably an orthopaedic surgical forceps, in a saline solution,
centrifuging the saline solution containing the fragmented bioptic sample, in order to obtain mesenchymal stromal stem cells.

The biopsy of the iliac crest of the pelvis is carried out by orthopaedic surgery, which guarantees the sterility of the sample from the moment that the bone is exposed by incision and there is no contact with the skin.

In other words, the bone stroma is obtained with an orthopaedic instrument larger than the needle and drill used for obtaining medullary (marrow) blood, such orthopaedic instrument being apt to extracting a bone biopsy with consequent minimisation of the presence of medullary blood.

Before introduction into the bone it is therefore necessary to cut the skin, a factor that further minimises the presence of epithelial cells and other possible contaminants. With this instrument a “carrot” of spongy bone is obtained, substantially free of liquid components. Such “carrot” of spongy bone contains bone cells (osteoblasts), a large quantity of mesenchymal stromal stem cells (about 500,000 mesenchymal stromal stem cells can be obtained from one bone carrot) and a small (CD34+) haematopoietic stem cell component. Blood cells, including lymphocytes, are nearly absent.

The advantages deriving from using bone stroma to obtain mesenchymal stem cells are therefore:
a greater quantity of mesenchymal stromal stem cells, which can be expanded in vitro to further increase their numbers, or used directly in therapy,
less contamination with CD34+ and other blood cells (macrophage, lymphocytes, etc.) and epithelial cells, which interfere negatively in vitro both with the adhesion process and with expansion, and on the therapeutic level with the immunomodulant function typical of mesenchymal stem cells.

After extraction the sample (“carrot” of spongy bone) is placed in a plate with saline solution and completely fragmented with surgical forceps. The solution in which the bone stroma was ground is aspirated and centrifuged. The pellet obtained is resuspended in culture media to then be transferred to a cell culture container where, in a period of 24 hours, adhesion of only mesenchymal stromal stem cells takes place, which in this way are selected.

As was briefly stated above, the advantage of such sampling with respect to a sample of medullary blood is that the presence of cells positive for haematopoietic (CD14, CD45 and CD34) and endothelial (CD31) markers is extremely reduced. Such cells—if present—are eliminated by means of washing with saline solution, since they do not adhere to the container.

The cellular population selected in this way may be used successively for cellular therapy of pathologies that can benefit from the administration of such mesenchymal stromal stem cells or from such mesenchymal stromal stem cells differentiated into a cellular phenotype of interest. The stem cells obtained thusly can, for example, be induced to differentiate into osteoblasts, myocytes, chondrocytes, neuroblasts, etc.

The various operations will now be described in detail.

a) Extraction of Stem Cells from Deep Bone Biopsies

The stem cells are obtained by means of minimally invasive orthopaedic surgery that consists of obtaining a bone biopsy sample from the iliac crest of the pelvis. The advantages of this type of sampling are mainly the sterility of the sample obtained (because the sample is obtained from bone that is exposed by means of an incision and so does not come in contact with the skin). Not being a blood sample, there is less contamination with CD34+ and endothelial cells, with respect to samples obtained with traditional sampling by means of aspiration.

The sample is placed in sterile tubes in saline solution.

The sample is removed from the container under a laminar flow hood and transferred to a petri dish together with 5 ml of saline solution.

The sample is held with long forceps and triturated lengthwise with orthopaedic surgical forceps until completely pulverised.

The solution in which the marrow is ground is aspirated, transferred into a centrifuge tube and centrifuged.

The resulting pellet is resuspended in culture medium and transferred to a 75 cm² cell culture flask.

The flask is transferred to an incubator with controlled CO₂ (5%), temperature (37° C.) and humidity (95%) for 24 hours (or longer, when cellular expansion is desired).

b) Selection and Detachment of the Mesenchymal Cell Population

Twenty four hours after seeding in the flask the cells are adherent to the culture surface, but have not yet undergone the first mitosis. Contaminating cells are eliminated by washing with saline solution pre-warmed to 37° C. and only adherent cells, that is, CD90+, CD31−, CD34−, CD45− cells, are maintained.

At this point, such cells are left in culture to increase in number through expansion or detached from the flask by means of enzymatic proteolysis, followed by introduction of 10 ml of culture medium into the flask to inhibit the action of trypsin (the culture medium must contain FBS, universally used as an inhibitor of the enzymatic reaction caused by trypsin on cell-cell and cell-substrate adhesion molecules) and they are transferred into centrifuge tubes. They are centrifuged, eliminating the supernatant and resuspending the resulting pellet in physiological solution.

The selection by adhesion of such population is a simple method suitable for the possible successive expansion and differentiation procedures.

Naturally, without prejudice to the underlying principle of the invention, the structural details and the embodiments may vary, even appreciably, with reference to what has been described by way of example only, without departing from the scope of the invention as defined by the annexed claims.

### BIBLIOGRAPHY

[1] Human bone marrow mesenchymal stem cells in vivo. E. Jones and D. McGonagle. Rheumatology 2008;47;126-131

[2] Bone marrow stromal cells: nature, biology, and potential applications. Bianco, P., Riminucci, M., Gronthos, S. and Robey, P G. Stem cells 2001; vol 19 no 3;180-192

[3] Multilineage potential of adult human mes-enchymal stem cells. Pittenger, M. F., A. M. Mackay, S. C. Beck, et al. Science 284: 143-147.

[4] Neurons Derived From Human Mesenchymal Stem Cells Show Synaptic Transmission and Can Be Induced to Produce the Neurotransmitter Substance P by Interleukin-1α. Kyung Jin Cho, Katarzyna A. Trzaska, Steven J. Greco, Joseph McArdle, Fu Shun Wang, Jiang-Hong Ye, Pranela Rameshwar Stem Cells. 2005;23:383-391.

[5] Mesenchymal stem cells: biological characteristics and potential clinical applications. Kassem M. Cloning Stem Cells. 2004;6(4):369-74.

1. Process for obtaining mesenchymal stromal stem cells comprising the following operations:
providing a bioptic sample of the iliac crest of the pelvis,
fragmenting said bioptic sample with a surgical forceps, preferably an orthopaedic surgical forceps, in a saline solution,
centrifuging said saline solution containing said fragmented bioptic sample, in order to obtain mesenchymal stromal stem cells.

2. Process for obtaining mesenchymal stromal stem cells according to claim 1, wherein said mesenchymal stromal stem cells obtained after said operation of centrifuging are suspended in a culture medium.

3. Process for obtaining mesenchymal stromal stem cells according to claim 2, wherein said mesenchymal stromal stem cells suspended in said culture medium are incubated in a container for at least 12 hours, preferably 18-36 hours, more preferably about 24 hours, in order to obtain said mesenchymal stromal stem cells adhered to the walls of said container.

4. Process for obtaining mesenchymal stromal stem cells according to claim 3, wherein said mesenchymal stromal stem cells adhered to the walls of said container are subjected to a washing operation, in order to obtain adhered mesenchymal stromal stem cells characterized by at least one of the following markers: CD90+; CD31−; CD34−; CD45−.

5. Process for obtaining adherent mesenchymal stromal stem cells according to claim 4, in which said mesenchymal stromal stem cells are characterized by the following markers: CD90+; CD31−; CD34−; CD45−.

6. Process for obtaining mesenchymal stromal stem cells according to claim 4, wherein said adhered mesenchymal stromal stem cells are subjected to an enzymatic proteolysis, in order to obtain mesenchymal stromal stem cells in suspension.
